(19) United States
(12) Patent Application Publication
(10) Pub. No.: US 2007/0225788 A1
(43) Pub. Date: Sep. 27, 2007

(54) Device and method for placing a stent at the ostium of a blood vessel

(76) Inventors: Robert E. Fischell, Dayton, MD (US); Tim A. Fischell, Kalamazoo, MI (US); Malcolm Foster, Knoxville, TN (US)

Correspondence Address: Dr. Robert E. Fischell, 14600 Viburnum Drive, Dayton, MD, 21036, US

(21) Appl. No.: 11/388,161

(22) Filed: Mar. 24, 2006

(51) Int. Cl. A61F 2/06

(52) U.S. Cl. 623/1.11

(57) ABSTRACT

A first aspect of the present invention is an ostial stent positioner that has the form of a wire for most of its length and having a cylinder with expandable legs situated at the positioner's distal end. The cylinder with its attached wire acts as an introducer sheath to introduce a stent delivery system with a stent into the artery that is to be stented. A second aspect of the present invention is a method for accurately placing a stent at the ostium of an artery that would have an ostial stenosis. Examples of such arteries that have ostial stenoses are the right and left main coronary arteries, a saphenous vein graft as used in coronary bypass surgery and the renal arteries. Each of these arteries has an ostium situated at the aorta.

### FIELD OF USE

This invention is in the field of devices for placing stents within a stenosis that extends to or near the ostium of an artery.

### BACKGROUND OF THE INVENTION

Although most stenoses do not occur at the ostium of an artery, there are thousands of cases each month where the mouth of an artery (the ostium) is substantially obstructed at its aortic take-off; this is called an aorto-ostial lesion. In such cases, the interventional cardiologist or radiologist is frequently unable to place the stent's proximal end within ±2 mm of the ostial plane. Two types of incorrect stent positions are (1) when the stent's proximal end extends more than 2 mm into the aorta, and (2) when the stent's proximal end is placed more than 1-2 mm into the artery distal to the ostial plane.

In U.S. Pat. No. 6,458,151, F. S. Saltiel describes an ostial stent positioning device. However, the most important feature of such a device; namely, and expandable distal portion that touches the wall of the aorta near the ostium of the artery to be stented is not optimized for easy usage of such a device.

In U.S. Pat. No. 5,749,890, A. Shaknovich utilizes a stent mounted on a catheter that has an inflatable section that touches the wall of the aorta in the vicinity of the ostium of the artery that is to be stented. Such a design precludes an accurate stent positioning system that can be used with the stent delivery system of any manufacturer.

### SUMMARY OF THE INVENTION

A first aspect of the present invention is an ostial stent positioner that has the form of a wire for most of its length and having a cylinder with expandable legs situated at the positioner's distal end. The cylinder with its attached wire acts as an introducer sheath to introduce a stent delivery system with a stent into the artery that is to be stented. A second aspect of the present invention is a method for accurately placing a stent at the ostium of an artery that would have an ostial stenosis. Examples of such arteries that have ostial stenoses are the right and left main coronary arteries, a saphenous vein graft as used in coronary bypass surgery and the renal arteries. Each of these arteries has an ostium situated at the aorta.

The method for using this invention would be to first back load the ostial stent positioner within a guiding catheter. The next action would be to place the guiding catheter through the aorta in a conventional manner so that its distal end will be engaged within or near the ostium of the artery that is to be stented. A guide wire would then be advanced through the guiding catheter until its distal end was placed distal to the stenosis. If pre-dilitation of the ostial stenosis was needed, a balloon angioplasty catheter would be advanced over the guide wire and through the guiding catheter and the catheter's balloon would be inflated to pre-dilate the stenosis. After the balloon angioplasty catheter was removed from the guiding catheter (or if no pre-dilatation was required) then a stent delivery system with the required stent would be advanced over the guide wire until the stent's proximal end lay distal to the ostium of the artery. The stent delivery system would have its proximal radiopaque marker band placed just distal to the ostial plane of the artery to be stented. While retaining the guide wire and a distal portion of the stent delivery system in the artery, the guiding catheter with the positioner inside would then be pulled back a short distance into the aorta. The positioner would then be advanced until its expandable legs at the positioner's distal end extended beyond the guiding catheter's distal end, thus allowing the expandable legs to fully expand. The guiding catheter would then be advanced until its distal end surface pushes gently against the positioner's expandable legs to engage them against the wall of the aorta and generally align the legs at the ostium of the artery that is to be stented. The plane of the “feet” which are located at the distal ends of the expandable legs would then be situated at the artery's ostial plane. Since the expandable legs would have feet that would be formed from a material that included a radiopaque substance or from a metal that is coated with or made from a radiopaque metal, the interventional cardiologist who is performing this procedure would have a clear angiographic/fluoroscopic marker of the ostial plane of the artery that is to have a stent placed within the ostial stenosis of that artery. The interventional cardiologist would then pull the stent delivery system back until the proximal radiopaque marker band within the balloon of the stent delivery system was aligned appropriately relative to the radiopaque feet of the expandable legs. The balloon would then be inflated to deliver the stent accurately at the ostial stenosis with the stent's proximal end lying within 2 mm of the ostial plane of the artery (typically just proximal to the true ostial plane). It is expected that an experienced interventional cardiologist could place the proximal end of the stent within 1.0 mm, just proximal to the ostial plane.

The main objects of this invention is to provide a means and method for accurately placing the proximal end of a stent within ±2 mm of the ostial plane of an artery that has a stenosis located at or near the ostium of that artery.

Another object of this invention is to place the proximal end of a stent within ±1.0 mm of the ostial plane of an artery that has a stenosis located at or near the artery's ostium.

Still another object of the present invention is to teach a method for accurately placing a stent within an ostial stenosis.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a side view of a Touhy-Borst fitting, a guiding catheter and an ostial stent positioner that acts as an introducer sheath for placing the proximal end of a stent in close proximity to the ostial plane of an artery that has an ostial stenosis.

FIG. 2 is a longitudinal cross section of a distal portion of the ostial stent positioner located within the guiding catheter showing the expandable legs in their folded state.

FIG. 3 is a cross section of a distal portion of the guiding catheter, a stent on a stent delivery system and the positioner showing the distal end plane of the feet of the expandable legs placed at the ostial plane of an artery having an ostial stenosis.

FIG. 4 is an alternate embodiment of the present invention using a self-expandable cylinder with expandable legs that is made from a shape memory alloy such as Nitinol.

FIG. 5 is a cross section of an introducer that is designed to facilitate the introduction of the self-expandable cylinder of FIG. 4 into a guiding catheter.

### DETAILED DESCRIPTION OF THE INVENTION

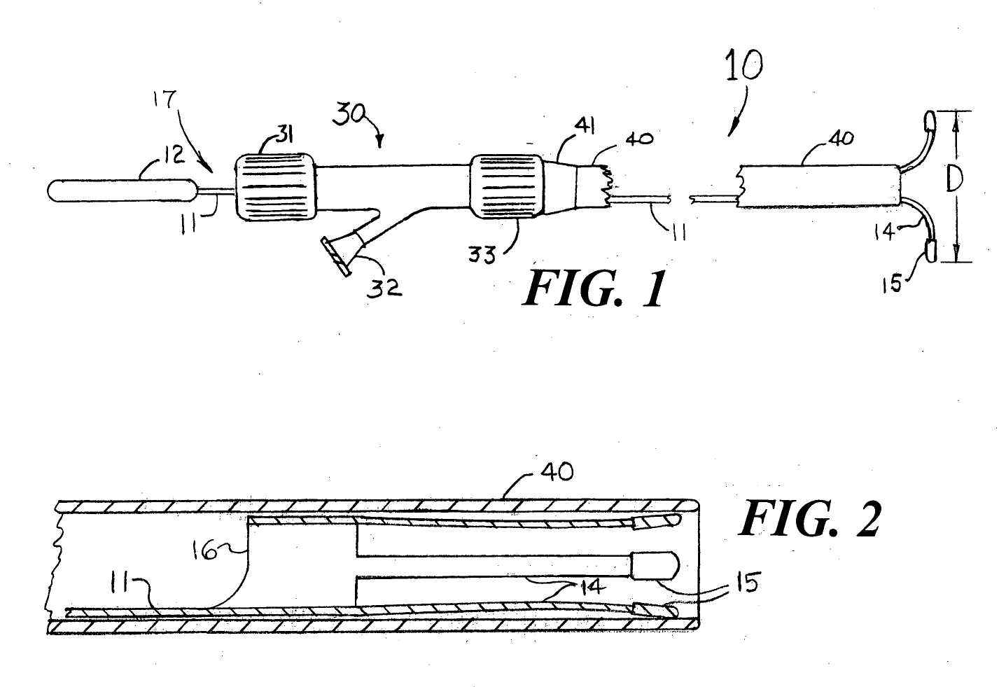

FIG. 1 is a side view of a catheter system 10 whose object is to accurately place a stent with its proximal end being situated close to the ostial plane of an artery having an ostial stenosis. The catheter system 10 would include an ostial stent positioner 17 that has a wire 11 which connects a small diameter handle 12 to a cylinder 16 (shown in FIG. 2) which has expandable distal end legs 14 with radiopaque feet 15. FIG. 1 also shows a guiding catheter 40 that has a proximal Luer fitting 41 that is joined to a Touhy-Borst fitting 30. When the feet 15 are fully expanded, the diameter “D” would typically be between 4 and 10 mm for coronary artery stenting and between 5 and 15 mm for stenting a renal artery. When the expandable legs 15 with radiopaque feet 15 are fully expanded they would have the general appearance of the petals of a flower. When the legs 15 are pushed forward beyond the distal end of the guiding catheter 40, they expand radially outward as shown in FIG. 1. When the handle 12 is pulled back, the legs 15 are retracted into the guiding catheter 40 and then the positioner 17 can be pulled out of the guiding catheter 40 after the stent has been placed into the ostial stenosis.

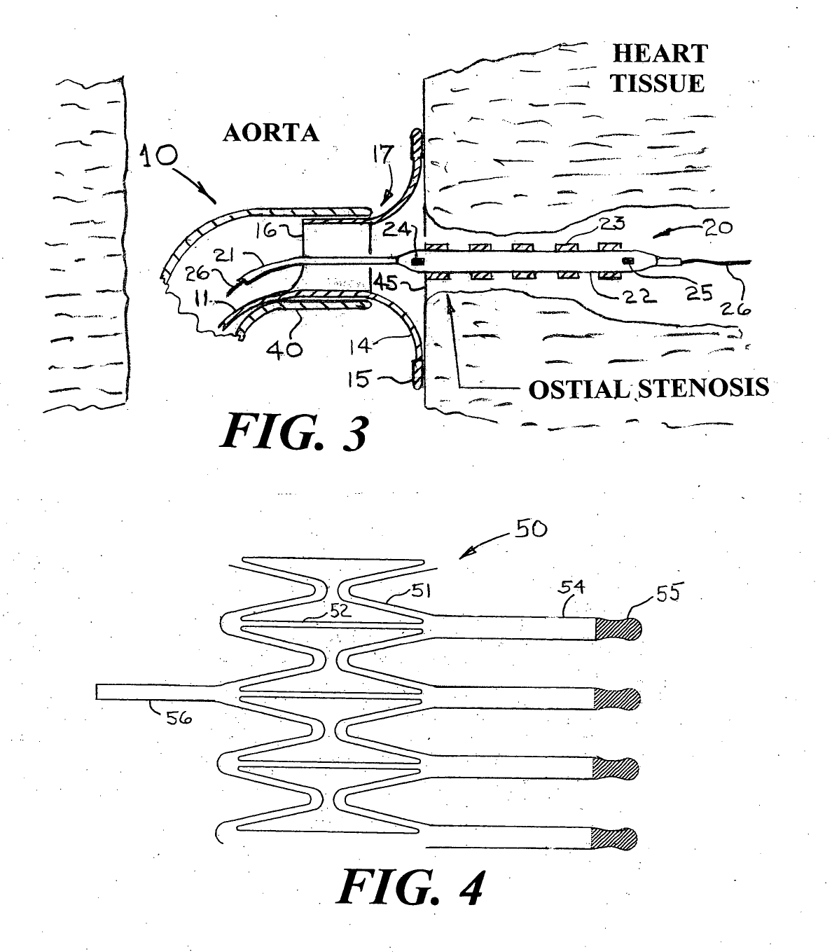

The Touhy-Borst fitting 30 has an adjustable seal fitting 31 (which is a hemostasis valve) that can initially be slightly loosened to allow the positioner 17 to be advanced or pulled back through the guiding catheter 40 without excessive blood leakage. When the expandable legs 14 are in their correct position for placement at the ostial plane of a stenosed artery, (as seen in FIG. 3) the adjustable seal fitting 31 can be tightened to hold a fixed position of the legs 23 relative to the guiding catheter 40 during stent deployment. The Luer fitting 32, being in fluid communication with the lumen of the guiding catheter 40, can be used for flushing the lumen with saline solution and/or for injecting contrast medium. The Luer connector 33 is used to form a removable fluidic seal with the Luer fitting 41 of the guiding catheter 40.

FIG. 2 is an enlarged cross section of the distal portions of the guiding catheter 40 and the positioner 17. The positioner 17 is shown with its expandable legs 14 in their unexpanded state within the guiding catheter 40. In this state, the guiding catheter 40 can be advanced through an introducer sheath at the patient's groin until its distal end is within the ostium of the artery that is to be stented. Furthermore, in this state, both a guide wire and a stent delivery system can be advanced through the guiding catheter 40 and through the ostial stenosis. The cylinder 16 is attached at its proximal end to the wire 11 and at its distal end to each of the four legs 14. Although 3 legs 14 (of an actual 4 legs) are shown in FIG. 2, as few as 2 or as many as 16 of petal-like legs 14 could be used for an effective array of expandable legs 14.

FIG. 3 is a cross section of a distal portion of the catheter system 10 shown with the distal plane 45 of the feet 15 placed at the ostial plane of a stenosed artery. The feet 15 are attached to the expandable legs 14 that are attached to the cylinder 16 which has its position within the guiding catheter 40 adjusted by means of the wire 11. Any such placement of the feet 15 can be defined as having their distal plane 45 “co-planar” with the ostial plane of the artery that has an ostial stenosis. FIG. 3 also shows a guide wire 26 placed through the stent delivery system 20 which has a shaft 21, a proximal radiopaque marker band 24, a distal radiopaque marker band 25 and a stent 23 mounted onto a balloon 22. The ostial stent positioner 17 would be designed to introduce essentially any commercially available stent delivery system 20 into an arterial stenosis. Thus, any interventional cardiologist could use the positioner 17 with any stent delivery system that he or she favors. FIG. 3 also shows how the guiding catheter 40 is used to gently push the feet 15 against the wall of the aorta at the ostium of the stenosed artery.

At the start of the stenting procedure, the ostial stent positioner 17 would be positioned as shown in FIG. 2 with the expandable legs 14 placed inside the guiding catheter 40. The catheter system 10 and the guide wire 11 could then be advanced through a conventional introducer sheath (not shown) typically placed at the groin of the patient into whom the stent 23 is to be placed. The guide wire 26 (or a separate 0.035 inch diameter guide wire) would be placed into and through the ostial stenosis and the guiding catheter 40 would be advanced until its distal tip was placed through the arterial ostium. The stent delivery system 20 would then be advanced over the guide wire 26 and through the guiding catheter 40 until the proximal radiopaque marker band 24 was positioned just distal to the ostium of the stenosed artery. The guiding catheter 40 would then be pulled back into the aorta. The positioner 17 (which was already back loaded into the guiding catheter 40) would then be advanced through the guiding catheter 40 until the expandable legs 14 extended out of the distal end of the guiding catheter 40. The guiding catheter 40 would then be pushed gently forward in a distal direction so as to obtain the configuration as generally shown in FIG. 3.

With the configuration as shown in FIG. 3, the interventional cardiologist would be able to clearly visualize the distal plane 45 of the radiopaque feet 15 and also visualize the proximal radiopaque marker band 24. When the radiopaque marker band 24 is pulled backward until it is co-planar with feet 15, then the proximal end of the stent 23 would be placed within ±2 mm of the plane of the ostium of the vessel which is to be stented. The balloon 22 would then be inflated to deliver the stent 23 into the ostial stenosis. Thus, an interventional cardiologist should be able to readily place the proximal end of the stent 23 within ±2 mm of the ostial plane. With some experience, it is expected that the proximal end of the stent 23 could be placed within at least ±1.0 mm of the ostial plane and probably within ±0.5 mm.

Although one method for accurately placing the stent 23 into an ostial stenosis has been described herein, it should be understood that there are several other ways that the present invention could be used to provide accurate stent positioning within an ostial stenosis. For example, the guiding catheter 40 with the positioner 17 in place as shown in FIG. 2 could first be placed over a 0.035 inch diameter guide wire and into the lumen of the ostial stenosis. That larger diameter guide wire could then be removed and a 0.014 inch diameter guide wire could be placed through the stenosis. The stent delivery system 20 could then be advanced over that guide wire 26 and positioned as shown in FIG. 3. The guiding catheter could then be pulled back and the expandable legs 14 could then be deployed as described herein. An important feature of the system 10 is that the guiding catheter 40 and positioner 17 could be held to be motionless while the guide wire 26 or the stent delivery system 20 could be advanced forward or pulled back to obtain an accurate positioning of the stent 23 within the ostial stenosis.

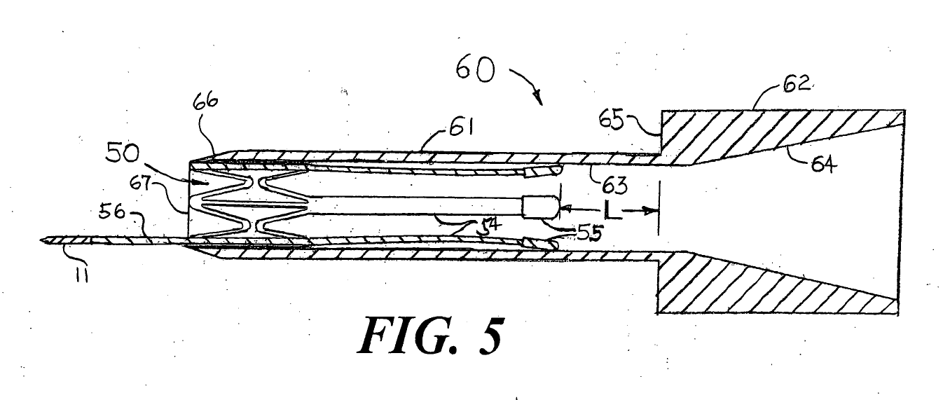

FIG. 4 is a flat layout view of self-expandable cylinder 50 that would replace the fixed diameter cylinder 16 of FIGS. 2 and 3. The strut 56 is designed to join to a wire that is equivalent to the wire 11 of FIGS. 1, 2 and 3, which wire is used to move the ostial stent positioner 17 within the guiding catheter 40. FIG. 5 shows the wire 11 attached to the proximal end of the strut 56. This attachment can be by means of welding, soldering or (with a somewhat different configuration) by means of a biocompatible adhesive. It is also conceived that the cylinder 50 and the wire 11 can be formed from a single piece of metal. The circumferential struts 51 provide a spring-like action to gently place the straight struts 52 against the inner wall of the guiding catheter 40. The expandable legs 54 with radiopaque feet 55 are joined to both the struts 51 and 52. The feet 55 could be made radiopaque by plating with a highly radiopaque metal such as platinum, gold or tantalum or they could be made from a high density metal.

To introduce a stent delivery system into a coronary artery, the typical diameter for the guiding catheter 40 would be 6, 7 or 8 French (Fr). It would be highly desirable for the ostial stent positioner 17 to be made with a single diameter of its cylinder that holds the expandable legs 54. This would decrease the inventory requirements for the positioner 17 for each cath lab that performs coronary interventions. Specifically, only one diameter of the expandable struts 51 would be required and it would fit into guiding catheters that are either 6, 7 or 8 Fr. It would also be highly desirable to have the cylinder 50 (as shown in FIG. 4) expand radially outward to gently press outwardly onto the inner surface of the guiding catheter 40. To that end, the cylinder 50 could be formed from a shape memory alloy such as Nitinol. To have a single product that would be suitable for 6, 7 or 8 Fr guiding catheters, the outer diameter of the cylinder 50 should be approximately the inside diameter of an 8 Fr guiding catheter. Such a cylinder 50 would then also fit snugly within either 6 Fr or 7 Fr guiding catheters. The wall thickness for the cylinder 50 would ideally be between 0.001 and 0.003 inches.

FIG. 5 is a highly enlarged cross section of an introducer 60 that would be used to place the self-expandable cylinder 50 into the guiding catheter 40. The introducer 60 would have an outer diameter of its cylindrical portion 61 that was just smaller in diameter than the inner diameter of the 6 Fr guiding catheter. With the assistance of its tapered end 66, the introducer 60 could then be placed into a 6, 7 or 8 Fr guiding catheter. To insert the ostial stent positioner 17 with the self-expandable cylinder 50 into the introducer 60, the wire 11 attached to the strut 56 would first be placed through the cone 64 and then it would be pulled through the interior cylindrical surface 63. The conical surface 64 would compress the self-expandable cylinder 50 to a small enough outside diameter to be able to be inserted into a 6 Fr (or even smaller diameter) guiding catheter. It is also envisioned that a carefully made cylinder 50 could be compressed enough to be placed into and used with a guiding catheter as small as 4 Fr. It should also be understood that a larger diameter guiding catheter 40 could be used specifically for treating an ostial stenosis in a renal artery. Guiding catheters as large as 14 Fr could be used for inserting a stent into an ostial stenosis. Of course, the expanded diameter of the cylinder 50 must also be at least slightly larger than the inside diameter of any such a guiding catheter. When the wire 11 has been used to pull the self-expandable cylinder 50 into the interior cylinder 63, the proximal end of the cylinder 50 should be generally aligned with the plane 67 of the introducer 60 as shown in FIG. 5. When this condition is met, the distal end of the radiopaque feet 15 will be a distance “L” from the plane of the shoulder 65 of the holding cylinder 62. This positioning will accurately align the feet 55 within the guiding catheter 40 so that the configuration will be as shown in FIG. 2.

To properly place the self-expandable cylinder 50 into the guiding catheter 40, the wire 11 is first back loaded through the guiding catheter 40 and the introducer 60 is pushed through the distal end of the guiding catheter 40 until the shoulder 65 is against the guiding catheter's distal end. The introducer 60 is then pulled out of the guiding catheter 40 while holding onto the handle 12 of FIG. 1 of the ostial stent positioner 17. When this is done, the distal end of the radiopaque feet 15 will be exactly the distance “L” from the distal end of the guiding catheter 40. This will be exactly the correct distance when the guiding catheter 40 including the ostial stent positioner 17 are advanced together until the distal end of the guiding catheter 40 lies at or into the ostium of the stenosed artery. The holding cylinder 62 is designed so that it can be easily held by the operator when placing the introducer 60 into the guiding catheter 40. The introducer 60 could be machined from a metal such as aluminum or molded from a clear plastic material such as polycarbonate. A transparent (i.e., a clear) plastic would have the advantage of allowing the operator to see the self-expandable cylinder 50 being advanced into the introducer 60 and the guiding catheter 40. Alternatively, the introducer 60 could be formed from a thin metal cylinder that makes up the cylinder 61 that is molded into the rest of the structure of the introducer 60.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. In combination a guiding catheter and an introducer sheath type of ostial stent positioner for facilitating the placement by an operator of the proximal end of a stent within ±2 mm of the ostial plane of an artery that has an ostial stenosis, the positioner having a wire extending for most of its length that is attached to a cylinder that is within the guiding catheter, the cylinder also being attached to expandable distal legs that end with radiopaque feet that form a distal plane when they are pushed in a distal direction out of the distal end of the guiding catheter and against the interior wall of the aorta, the positioner being designed to be placed within the guiding catheter and being further designed for placement of the distal plane of the radiopaque feet to be substantially co-planar with the ostial plane of the artery when the guiding catheter is urged forward in a distal direction after the expandable distal legs have been expanded radially outward.

2. The combination of claim 1 where the guiding catheter has a diameter that lies between 4 Fr and 14 Fr.

3. The combination of claim 1 where the wire has a handle at its proximal end to facilitate the handling of the positioner by the operator.

4. The combination of claim 3 where the handle is formed from a plastic material.

5. The combination of claim 1 where the expandable legs are formed from a radiopaque metal or coated with a radiopaque metal.

6. The combination of claim 5 where the feet on the expandable legs have an outside diameter when expanded that lies between 4 and 15 mm.

7. The combination of claim 1 where the positioner has expandable legs that are formed from a combination of a plastic material and a metal, the combination being generally radiopaque.

8. The combination of claim 1 where the positioner has a collection of expandable legs which collection of legs is generally in the shape of a flower that has at least two petals.

9. The combination of claim 8 where the positioner's expandable legs have at least four petals.

10. The combination of claim 1 where the cylinder that is attached to the expandable legs is of a fixed diameter.

11. The combination of claim 1 where the cylinder that is attached to the expandable legs is self-expandable.

12. The combination of claim 11 where the self-expandable cylinder that is attached to the expandable legs is formed from shape memory alloy Nitinol.

13. A method for placing the proximal end of a stent within an artery that has an ostial stenosis so that the stent's proximal end is positioned within ±2 mm of the artery's ostial plane, the method including the following steps:
a) advancing an introducer sheath type of positioner that has an expandable distal legs within a guiding catheter with the expandable legs in an unexpanded state;
b) causing the expandable legs to expand in a region beyond the distal end of the guiding catheter;
c) urging the guiding catheter in a forward, distal direction so that a distal plane of the expanded legs is placed substantially co-planar with the ostial plane of the artery that has the ostial stenosis; and
d) positioning a stent within the stenosis of the artery so that the stent's proximal radiopaque marker band is situated relative to the distal plane of the expanded legs of the positioner so that the stent's proximal end is located within ±2 mm of the ostial plane when the stent is deployed.

14. The method of claim 13 where the positioner's expanded legs are generally shaped in the form of a flower with at least two petals.

15. The method of claim 13 where the expanded legs have at least some portion that is formed from a radiopaque metal or coated with a radiopaque metal.

16. The method of claim 13 where the expanded legs are attached to a self-expandable cylinder that is formed from Nitinol.

17. The method of claim 16 where the expanded legs and the self-expandable cylinder are both formed from Nitinol.
